(19) United States
(12) Patent Application Publication
(10) Pub. No.: US 2026/0263657 A1
(43) Pub. Date: Sep. 10, 2026

(54) METHODS FOR FABRICATING A REGENERATIVE MATERIAL FROM EXTRACTED TEETH

(71) Applicant: Ventas Bio Pte. Ltd., Singapore (SG)

(72) Inventors: Kim Lung Henry Ho, Singapore (SG); Dih Hann Simon Ng, Singapore (SG)

(21) Appl. No.: 19/166,642

(22) PCT Filed: Apr. 18, 2024
(86) PCT No.: PCT/SG2024/050254

§ 371 Date: Sep. 18, 2025

(30) Foreign Application Priority Data
Apr. 18, 2023 (SG) 10202301081W

(51) Int. Cl. A61L 27/36

(52) U.S. Cl. CPC A61L 27/3608; A61L 27/3687; A61L 27/3691; A61L 2430/02

(57) ABSTRACT

The present disclosure relates to methods for processing extracted teeth into regenerative material. Specifically, the root portion of the extracted teeth is processed to obtain the regenerative material. Use of the regenerative material for the regeneration of periodontal 5 tissue around teeth that have been lost due to injury or periodontal disease is also described herein.

### FIELD OF THE DISCLOSURE

The present disclosure relates to a method for manufacturing regenerative material, in particular to methods for manufacturing regenerative materials from extracted teeth.

### BACKGROUND

Periodontal disease is a common condition that results in the loss of the supporting tissue and bone around the teeth. If left untreated, this disease may cause the teeth to become loose and eventually fall out or require extraction. Current treatment options for periodontal disease include scaling and root planning as well as surgical procedures such as Guided Tissue Regeneration (GTR), bone grafting and soft tissue grafting. While these treatments can be effective, the above treatments do not always provide predictable outcomes. For instance, bone grafting and soft tissue grafting pose risks of rejection. Further, the use of autogenic tissue would mean that it is necessary to harvest autogenic tissue by means of a secondary surgery, resulting in a longer and possibly more complex surgical procedure. The secondary surgery to harvest autogenic tissue would also result in higher overall costs. Further, challenges in relation to loss of jawbone, which usually requires regenerating to place dental implant remain unaddressed. Additionally, the challenges surrounding jawbone deterioration and the need for restorative measures, such as bone grafting regeneration, to facilitate dental implant procedures have yet to be adequately addressed. Furthermore, the difficulties in effectively regenerating resorbed jawbone to enable successful dental implant procedures remain insufficiently resolved.

In view of the foregoing, therefore there is a need to provide methods that overcome or at least ameliorate the above limitations. More importantly, the current disclosure may provide an alternative treatment option that is more effective, predictable and less complex.

### SUMMARY

There is provided a method of fabricating a graft material from a tooth. In accordance with some embodiments of the disclosure, said method include the steps of providing one or more root portion of the tooth, contacting the one or more root portion of the tooth with a first reagent at a predefined temperature for a predefined duration so as to disinfect the one or more root portion of the tooth and sterilizing the disinfected one or more root portion of the tooth at a predefined sterilization temperature and pressure, thereby obtaining said graft material.

Optionally, the method may further comprise reducing particle size of the sterilized one or more root portion of the tooth thereby obtaining said graft material. The reduced particle size of the one or more root portion of the tooth may be in a range from 100 μm to about 5,000 μm.

According to some embodiments of the disclosure, the first reagent may comprise one or more oxidizing agents containing chlorine and oxygen. Optionally, the oxidizing agent may comprise sodium hypochlorite or calcium hypochlorite and optionally may further comprise sodium hydroxide. Optionally, the first reagent may be a base including but not limited to NaOH, KOH, Ca(OH)₂, Mg(OH)₂ and Ba(OH)₂.

According to some embodiments of the disclosure, the predefined sterilization temperature may be from about 110° C. to about 150° C. and the predefined sterilization pressure may be from about 10 psi to about 50 psi. According to some embodiments of the disclosure, the graft material obtained from said method or process may be a tissue graft material or a bone graft material.

According to some embodiments of the disclosure, the disinfection of the one or more root portion may further comprise contacting one or more root with a second reagent. The second reagent may optionally be an oxidizing agent comprising a compound having two oxygen atoms. Optionally, the two oxygen atoms may be linked via a single covalent bond. The compound may be a peroxide selected from the group consisting of hydrogen peroxide, peroxy acid, metal peroxide and organic peroxide.

According to some embodiments of the disclosure, the method may further include disintegrating one or more cells from the one or more root portion of the tooth by contacting the one or more root portion of the tooth with a second oxidizing agent under ultrasonic vibration. Optionally, the method may further include washing the decellularized one or more root portion of the tooth with one or more acid with a predefined ratio between volume of the one or more acid used and volume of the portion of the tooth. The predefined volume ratio may be in a range from 1:2 to 20:1.

According to some embodiments of the disclosure, the method may further include lyophilizing the decellularized one or more root portion of the tooth under a predefined lyophilization condition.

According to some embodiments of the disclosure, the method may further include a second sterilization step. The second sterilization step may be undertaken using one of the following gases: ethylene oxide gas, vaporized hydrogen peroxide, chlorine dioxide gas, vaporized peracetic acid and nitrogen dioxide. Optionally, the second sterilization step may be undertaken using a gamma sterilization or an electron beam sterilization.

Further, there is provided a graft material from a tooth obtained by a method described herein. Said tooth may be a human tooth or an animal tooth. Optionally, the graft material obtained by the method above may comprise 20% (v/v) or more of octa calcium phosphate.

Additionally, in another aspect, there is provided a graft material from a tooth comprising 20% (v/v) or more of octa calcium phosphate.

Still further, there is provided a method of regenerating bone or tissue in a human comprising implanting a graft material obtained by a process described herein to a damaged or loss portion of the bone or tissue. The damaged or loss portion of the bone or tissue may be caused by a disease selected from the group consisting of periodontal discase, osteoarthritis, autoimmune diseases (including but not limited to rheumatoid arthritis, Sjogren's syndrome and lupus), osteoporosis, infection, tumors, developmental or congenital disorders, bone cancer, and bone disease. The damaged or loss portion of the bone or tissue may also be caused by trauma or injury, tooth extraction, ill-fitting dentures, bruxism, sinus issues, radiation therapy, and medication (for example administration of bisphosphonates). The periodontal disease may be selected from the group consisting of gingivitis, periodontitis, aggressive periodontitis, chronic periodontitis, systemic periodontitis and necrotizing periodontal disease. Advantageously, the sterilization step including sterilization using steam and sterilization using ethylene oxide or via radiation may eliminate immunogenicity thereby eliminating or at list minimizing the risk of rejection.

Yet advantageously, the method described in the present disclosure may retain bioactive materials and/or bioactive molecules in the graft material. The presence of bioactive materials or molecules including bioactive proteins in the graft material is useful as these proteins may promote regeneration of tissue and bone, in particular periodontal tissue and bone, when the graft materials are used in tissue and bone regeneration.

Still advantageously, the method for producing the graft material described herein may maintain the mechanical strength of the graft material. Hence, the materials may withstand mechanical load and preserve the space for the newly formed tissue and bone.

When used in tissue and/or bone regeneration or repair, the graft material obtained by the method described herein advantageously may regenerate periodontal tissues and/or bones including alveolar bone, periodontal ligament tissue and cementum. Since the structure and function of the periodontal tissue surround teeth has been lost due to periodontal disease or injury, tissue and/or bone regeneration by the graft material described herein may restore the periodontal tissue support of the teeth and preserve the function of the teeth.

Further advantageously, the graft material disclosed herein may be used without additional carrier or membrane. This is beneficial as it simplifies the surgical procedure by reducing the complexity as well as minimizing or eliminating the need for additional carrier or membrane. In turn, this would result in reducing surgical time and costs.

Additionally, the graft material obtained by the method disclosed herein may be suitable for allogenic use in humans. Since the allogenic tissue can be readily available, the need for the patient to undergo additional surgery to harvest autogenic tissue may be eliminated or minimized, thus reducing overall surgical time and costs. Hence, additional risks and morbidity may be avoided.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a method for fabricating a graft material, according to some embodiment of the disclosure.

### DETAILED DESCRIPTION

The present disclosure provides a method or process for fabricating graft material. Throughout the present disclosure, the terms “graft material” and “grafting material” have the same meaning and are used interchangeably.

In some embodiments, the graft materials or grafting materials described herein may be used in surgical procedures to replace or repair damaged tissue or bone. As used herein, a graft refers to a piece of tissue or material that is surgically implanted to replace missing or damaged tissue or bone. In some embodiments, the graft material may be autogenic as they are harvested from the subject's own body for example skin tissue, bone, or tendon. In some embodiments, the graft material may be allogenic as they are obtained from a donor of the same species. Examples of the allogenic graft material include human skin tissue or bone. In some embodiments, the graft material may be xenogeneic as they are obtained from a different species. Non-limiting examples of the xenogeneic graft materials include pig skin and pig teeth.

In some embodiments, the method described herein may require a tooth as a starting material. In some embodiments, the tooth may be human or animal tooth.

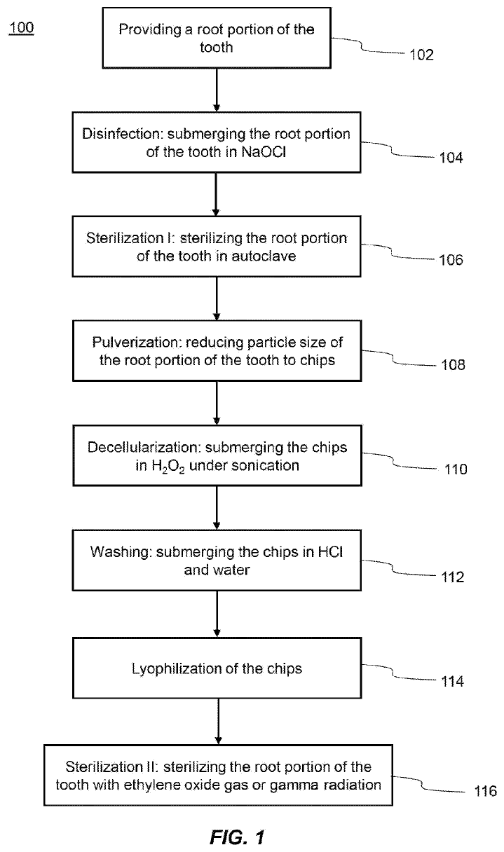

In some embodiments, as can be seen from FIG. 1, the method may comprise providing one or more root portion of the tooth (102). In some embodiments, the one or more root portion of the tooth may be obtained by removing crown portion of the human extracted tooth. It is to be appreciated that in the method disclosed herein, the tooth may generally comprise root portion and crown portion. In some embodiments, the root portion may be an individual or a multiple root portion. In some embodiments, the removing crown portion may comprise cutting the tooth, specifically cutting cemento-enamel junction (CEJ). As used herein, CEJ, also known as “cervical line” refers to the natural boundary or junction between the enamel of the tooth and the cementum of the root portion. CEJ is the only zone or area where the enamel and cementum are in contact with each other. In some embodiments, cutting CEJ comprises cutting at a region proximate to the actual CEJ. In some embodiments, the cutting may be performed using a cutting device. Non-limiting examples of such cutting device include diamond impregnated burs or discs. In some embodiments, the cutting may be performed under water irrigation. In some embodiments, the cutting may be performed under Type I water, Type II water or water for injection irrigation.

Following the cutting step described above, the crown portion may be discarded as only the root portion will be used in a further process. For the case of multiple root portion such as those of molars, the root may be separated or divided into individual root portions by cutting the point where the multiple roots are joined together. In some embodiments, the cutting may comprise cutting furcation to afford the individual root portions. Similar as above, the cutting may be performed by using a cutting device. Non-limiting examples of such cutting device include diamond impregnated burs or discs. In some embodiments, the cutting may be performed under water irrigation. In some embodiments, the cutting may be performed under Type I water, Type II water or water for injection irrigation.

After the individual root portions are obtained, the next step is to disinfect the root portions (104). In some embodiments, the disinfecting may comprise contacting the one or more root portion of the tooth with a first reagent at a predefined temperature for a predefined duration so as to disinfect the one or more root portion of the tooth. In some embodiments, the contacting may comprise submerging the root portion in the first reagent. In some embodiments, the contacting may comprise submerging the root portion in the first reagent under ultrasound. It is to be appreciated that other stirring or agitation method (for example the use of shaker or magnetic stirrer) may be used and is not limited to ultrasonic stirring or ultrasonic agitation. The frequency of the ultrasound used may be in the range from 10 to 60 kHz.

In some embodiments, the first reagent may be one or more oxidizing agents containing chlorine and oxygen. In some embodiments, the oxidizing agent may be sodium hypochlorite, calcium hypochlorite or mixtures thereof. In some embodiments, the concentration of the oxidizing agent may be in the range of 1%-10%, for example 1%, 2%, 3%, 4%, 5%, 6%, 7%, 8%, 9%, 10%. In some embodiments, the concentration may be expressed in % weight (w/w) or % volume (v/v). In some embodiments, the concentration of the oxidizing agent may be in the range of 1% weight-10% weight, for example 2% weight-8% weight, 2.5% weight 6% weight, 4% weight-6% weight or 4.75% weight-5.75% weight.

In some embodiments, the first reagent may be a base. In some embodiments, the base may be a strong base or a weak base. In some embodiments, the base is selected from the group consisting of NaOH, KOH, Sr(OH)₂, Ba(OH)₂ and Ca(OH)₂. In some embodiments, the concentration of the base is in a range from about 0.05 M to about 4 M for example 0.05 M, 0.06 M, 0.08 M, 0.1 M, 0.15 M, 0.2 M, 0.5 M, 1 M, 1.5 M, 2 M, 2.5 M, 3 M, 3.5 M, or 4 M.

In some embodiments, the first reagent may be a mixture of one or more oxidizing agents containing chlorine and oxygen and a base. In some embodiments, the first reagent is a mixture of sodium hypochlorite and sodium hydroxide. In some embodiments, the first reagent is a mixture of calcium hypochlorite and sodium hydroxide.

In some embodiments, the contacting with the first reagent may be undertaken for a duration of from about 10 minutes to about 120 minutes for example 20 minutes, 30 minutes, 40 minutes, 50 minutes, 55 minutes, 60 minutes, 65 minutes, 70 minutes, 80 minutes, 90 minutes, 100 minutes, or 110 minutes. In some embodiments, the duration of the disinfection is from about 30 minutes to about 90 minutes. In some embodiments, the duration of the disinfection is from about 55 minutes to about 65 minutes.

In some embodiments, contacting with the first reagent may be undertaken in a predefined temperature in the range from about 20° C. to about 60° C. for example about 25° C., about 30° C. about 35° C., about 40° C., about 45° C., about 50° C., or about 55° C. In some embodiments, the temperature does not exceed 60° C. It is to be appreciated that the predefined temperature may be constant throughout the contacting step or it may be varied.

In some embodiments, following the contacting the root portion with or submerging in the first reagent, the root portion may be optionally washed in water. In some embodiments, the water may be Type I water, Type II water or water for injection. In some embodiments, the washing may be undertaken at a temperature in a range from about 20° C. to about 60° C. for example about 25° C., about 30° C., about 35° C., about 40° C., about 45° C., about 50° C., or about 55° C. In some embodiments, the temperature does not exceed 60° C. It is to be appreciated that the predefined temperature may be constant throughout the contacting step or it may be varied.

In some embodiments, the washing with water may be undertaken for a duration of from about 1 minute to about 20 minutes, for example 2 minutes, 3 minutes, 4 minutes, 5 minutes, 6 minutes, 10 minutes, 12 minutes, or 15 minutes.

In some embodiments, following the washing with water, the root portion may be optionally contacted with a second reagent for example by submerging the root portion in the second reagent. In some embodiments, the second reagent is an oxidizing agent comprising a compound having two oxygen atoms, the two oxygen atoms are linked via a single covalent bond. In some embodiments, the second reagent is a peroxide selected from the group consisting of hydrogen peroxide, peroxy acid, metal peroxide and organic peroxide. In some embodiments, the second reagent is hydrogen peroxide. In some embodiments, other suitable oxidizing agents may be used.

In some embodiments, the concentration of the hydrogen peroxide or the oxidizing agent may be in the range of 1%-10%, for example 1%, 2%, 3%, 4%, 5%, 6%, 7%, 8%, 9%, 10%. In some embodiments, the concentration may be expressed in % weight (w/w) or % volume (v/v). In some embodiments, the concentration of the oxidizing agent may be in the range of 1% weight-10% weight, for example 3% weight-8% weight, 3% weight-6% weight, 3% weight-4% weight or 4% weight-8% weight. In some embodiments, the contacting may comprise submerging the root portion in the second reagent under ultrasound. It is to be appreciated that other stirring or agitation methods (for example the use of shaker or magnetic stirrer) may be used and is not limited to ultrasonic stirring or ultrasonic agitation. The frequency of the ultrasound used may be in the range from 10 to 60 kHz.

In some embodiments, the contacting step with the second reagent or the oxidizing agent may be undertaken at a temperature in a range from about 20° C. to about 60° C. for example about 25° C., about 30° C., about 35° C., about 40° C., about 45° C., about 50° C., or about 55° C. In some embodiments, the temperature does not exceed 60° C. It is to be appreciated that the predefined temperature may be constant throughout the contacting step or it may be varied.

In some embodiments, the contacting with the second reagent or the oxidizing agent may be undertaken for a duration of from about 20 minutes to about 120 minutes for example 20 minutes, 30 minutes, 40 minutes, 50 minutes, 55 minutes, 65 minutes, 70 minutes, 75 minutes, 80 minutes, 90 minutes, 100 minutes or 110 minutes.

In some embodiments, the second reagent may be identical to the first reagent. In such an embodiment, the contacting step with the first reagent is repeated or undertaken in more than one cycle. In some embodiments, the disinfecting step may comprise contacting the root portion with the first reagent, where the first reagent is NaOCl₄, followed by washing in water. In some embodiments, the disinfecting step may further include contacting the root portion with the second reagent, where the second reagent is NaOCl₄. In some embodiments, the second reagent may be a base. In some embodiments, the base may be a strong base or a weak base. In some embodiments, the base is selected from the group consisting of NaOH, KOH, Sr(OH)₂, Ba(OH)₂ and Ca(OH)₂. In some embodiments, the concentration of the base is in a range from about 0.05 M to about 4 M for example 0.05 M, 0.06 M, 0.08 M, 0.1 M, 0.15 M, 0.2 M, 0.5 M, 1 M, 1.5 M, 2 M, 2.5 M, 3 M, 3.5 M, or 4 M.

In some embodiments, the second reagent is a mixture of one or more oxidizing agents containing chlorine and oxygen and a base. In some embodiments, the second reagent is a mixture of sodium hypochlorite and sodium hydroxide.

In some embodiments, following the disinfection step (104), the disinfected root portion may undergo a cleaning step to substantially remove adhered debris on the root portion. In some embodiments, the cleaning may be undertaken using diamond impregnated burs or tungsten carbide burs.

In some embodiments, the cleaning step may include washing the root portion in water or other suitable solvent. In some embodiments, the water may be Type I water, Type II water or water for injection. In some embodiments, the washing may be undertaken at a temperature in a range from about 20° C. to about 60° C. for example about 25° C., about 30° C., about 35° C., about 40° C., about 45° C., about 50° C., or about 55° C. In some embodiments, the washing temperature does not exceed 60° C. It is to be appreciated that the washing temperature may be constant throughout the contacting step or it may be varied. In some embodiments, the washing is undertaken under ultrasound. It is to be appreciated that other stirring or agitation method (for example the use of shaker or magnetic stirrer) may be used and is not limited to ultrasonic stirring or ultrasonic agitation. The frequency of the ultrasound used may be in the range from 10 to 60 kHz.

In some embodiments, the washing with water may be undertaken for a duration of from about 1 minute to about 20 minutes, for example 2 minutes, 3 minutes, 4 minutes, 5 in 5 minutes, 6 minutes, 10 minutes, 12 minutes, or 15 minutes.

In some embodiments, as can be seen from FIG. 1, the method may continue with sterilization (106) of the disinfected root portion. In some embodiments, sterilizing the disinfected one or more root portion of the tooth may be undertaken at a predefined sterilization temperature and pressure. In some embodiments, the sterilization may be undertaken in an enclosed reactor. In some embodiments, the enclosed reactor is an autoclave.

In some embodiments, the predefined sterilization temperature is from about 110° C. to about 150° C., for example 112° C., 115° C., 118° C., 120° C., 121° C., 122° C., 124° C., 126° C., 130° C., 132° C., 134° C., 135° C., 138° C., 140° C., 145° C. or 150° C. In some embodiments, the predefined sterilization temperature is from about 121° C. to about 134° C. In some embodiments, the predefined sterilization temperature is about 121° C. In some embodiments, the predefined sterilization temperature is not less than 121° C.

In some embodiments, the predefined sterilization pressure is from about 10 psi to about 50 psi, for example about 12 psi, about 15 psi, about 18 psi, about 20 psi, about 22 psi, about 25 psi, about 30 psi, about 35 psi, about 40 psi, about 45 psi or about 50 psi. In some embodiments, the predefined sterilization pressure is from about 15 psi to about 30 psi. In some embodiments, the predefined sterilization pressure is about 15 psi.

In some embodiments, the sterilization may be undertaken for a duration of from about 10 minutes to about 120 minutes for example 20 minutes, 30 minutes, 40 minutes, 50 minutes, 55 minutes, 60 minutes, 65 minutes, 70 minutes, 80 minutes, 90 minutes, 100 minutes, or 110 minutes. In some embodiments, the duration of the sterilization is from about 10 minutes to about 60 minutes. In some embodiments, the duration of the sterilization is about 30 minutes.

In some embodiments, the disinfected root portion may be autoclaved at temperature of about 121° C. to about 134° C., under 15 psi to 30 psi for about 30 minutes to 60 minutes. In some embodiments, the disinfected root portion may be autoclaved at about 121° C., under 15 psi for about 30 minutes.

In some embodiments, the sterilization described above (106) may be considered as a first sterilization. In some embodiments, the first sterilization may be undertaken more than one cycle or repeated as necessary. In some embodiments, the first sterilization is undertaken in one cycle.

In some embodiments, method (100) may further comprise, after the sterilization step, reducing particle size of the sterilized one or more root portion of the tooth thereby obtaining said graft material (108). In some embodiments, the reducing particle may be termed “pulverization”. It is to be appreciated that any suitable apparatus or method for reducing particle size may be used. In some embodiments, the reducing particle size may be by virtue of milling. It is to be understood that milling may involve grinding, cutting, or shaping of the root portion using a machine. A person skilled in the art will understand that the milling process can be used to create a variety of shapes, from simple flat surfaces to complex three-dimensional structures.

In some embodiments, when the root portion is subjected to a particle size reduction process, a smaller size of root portion particle may be formed. Depending on the parameters used, the reduced particle size may follow homogeneous or heterogeneous particle size distribution. In some embodiments, the milling process may involve feeding the root portion into a cutting tool, which rotates at high speed and removes material from the workpiece. In some embodiments, the cutting tool may be a single-point cutting tool, such as a drill bit, or a multi-point cutting tool, such as an end mill or a milling cutter. In some embodiments, various types of milling machines, including but not limited to vertical and horizontal milling machines, which differ in the orientation of the cutting tool, may be used. In a vertical milling machine, the cutting tool is mounted vertically and the workpiece is held in a horizontal position. In a horizontal milling machine, the cutting tool is mounted horizontally and the workpiece is held in a vertical position. In some embodiments, the particle size reduction step (108) may be undertaken more than one cycle or repeated as necessary until the desired particle size is achieved.

In some embodiments, the reduced particle size of the one or more root portion of the tooth is in a range from about 50 μm to about 5,000 μm for example 50 μm, 60 μm, 80 μm, 100 μm, 120 μm, 140 μm, 150 μm, 160 μm, 180 μm, 200 μm, 250 μm, 500 μm, 750 μm, 1,000 μm, 1,250 μm, 1,500 μm, 1,600 μm, 1,800 μm, 2,000 μm, 2,200 μm, 2,500 μm, 2,800 μm, 3,000 μm, 3,200 μm, 3,500 μm, 3,800 μm, 4,000 μm, 4,500 μm or 5,000 μm. In some embodiments, the reduced particle size of the one or more root portion of the tooth is in a range from about 150 μm to about 3,000 μm.

In some embodiments, method (100) may further comprise disintegrating one or more cells from the one or more root portion of the tooth (110) by contacting the one or more root portion of the tooth with a third reagent under ultrasonic vibration or ultrasonic energy In some embodiments, the third reagent is a second oxidizing agent. In some embodiments, the term “disintegrating” one or more cells refers to lysis. In some embodiments, lysis comprises rupturing a cell wall or a membrane that results in disintegration of the one or more cells.

In some embodiments, following the washing with water, the root portion may be optionally contacted with a second reagent for example by submerging the root portion in the second reagent. In some embodiments, the second reagent is an oxidizing agent comprising a compound having two oxygen atoms, the two oxygen atoms are linked via a single covalent bond. In some embodiments, the second reagent is a peroxide selected from the group consisting of hydrogen peroxide, peroxy acid, metal peroxide and organic peroxide. In some embodiments, the second reagent is hydrogen peroxide. In some embodiments, other suitable oxidizing agents may be used.

In some embodiments, the concentration of the hydrogen peroxide or the oxidizing agent may be in the range of 1%-10%, for example 1%, 2%, 3%, 4%, 5%, 6%, 7%, 8%, 9%, 10%. In some embodiments, the concentration may be expressed in % weight (w/w) or % volume (v/v). In some embodiments, the concentration of the oxidizing agent may be in the range of 1% weight-10% weight, for example 3% weight-8% weight, 3% weight-6% weight, 3% weight-4% weight or 4% weight-8% weight. In some embodiments, the contacting may comprise submerging the root portion in the second reagent under ultrasound. The frequency of the ultrasound used may be in the range from 10 to 60 kHz.

In some embodiments, the contacting step with the second reagent or the oxidizing agent may be undertaken at a temperature in a range from about 20° C. to about 60° C. for example about 25° C., about 30° C., about 35° C., about 40° C., about 45° C., about 50° C., or about 55° C. In some embodiments, the temperature does not exceed 60° C. It is to be appreciated that the predefined temperature may be constant throughout the contacting step or it may be varied.

In some embodiments, the contacting with the second reagent or the oxidizing agent may be undertaken for a duration of from about 20 minutes to about 120 minutes for example 20 minutes, 30 minutes, 40 minutes, 50 minutes, 55 minutes, 65 minutes, 70 minutes, 75 minutes, 80 minutes, 90 minutes, 100 minutes or 110 minutes. In some embodiments, the temperature of the contacting with the second reagent or the oxidizing agent does not exceed 60° C.

In some embodiments, the step (110) may be performed by contacting the one or more root portion of the tooth with a second oxidizing agent following a predefined volume ratio. In some embodiments, the ratio is a ratio between the volume of the second oxidizing agent and the volume of the chips. In some embodiments, the ratio is from about 1:2 to about 20:1 for example 1:1, 2:1, 3:1, 4:1, 5:1, 6:1, 7:1, 8:1, 10:1, 12:1, 15:1 and 20:1. In some embodiments, the ratio is about 4:1.

Following the contacting step above, step (110) may include a cleaning step. In some embodiments, the cleaning step may include washing the root portion in water or other suitable solvent. In some embodiments, the water may be Type I water, Type II water or water for injection. In some embodiments, the washing may be undertaken at a temperature in a range from about 20° C. to about 60° C. for example about 25° C., about 30° C., about 35° C., about 40° C., about 45° C., about 50° C., or about 55° C. In some embodiments, the washing temperature docs not exceed 60° C. It is to be appreciated that the washing temperature may be constant throughout the contacting step or it may be varied. In some embodiments, the washing is undertaken under ultrasound. The frequency of the ultrasound used may be in the range from 10 to 60 kHz.

In some embodiments, the washing with water may be undertaken for a duration of from about 1 minute to about 20 minutes, for example 2 minutes, 3 minutes, 4 minutes, 5 minutes, 6 minutes, 10 minutes, 12 minutes, or 15 minutes.

In some embodiments, the cleaning step may be performed by adjusting a ratio between the volume of the washing solution and the volume of the chips. In some embodiments, the ratio is from about 1:2 to about 20:1 for example 1:1, 2:1, 3:1, 4:1, 5:1, 6:1, 7:1, 8:1, 10:1, 12:1, 15:1 and 20:1. In some embodiments, the ratio is about 4:1.

In some embodiments, as can be seen from FIG. 1, the decellularized chips may undergo washing step (112). In some embodiments, the washing step may include submerging the chips in a solution containing one or more acids. In some embodiments, the one or more acids may be inorganic or organic acid. In some embodiments, the acid is hydrochloric acid (HCl). In some embodiments, concentration of the acid may be in the range of from about 0.01 M to about 1.5 M for example 0.1 M, 0.2 M, 0.5 M, 0.6 M, 0.7 M, 0.8 M, 1 M, 1.1 M, 1.3 M and 1.5 M. in some embodiments, the submersion in acid is undertaken under stirring condition and at a predefined temperature. In some embodiments, the submersion in acid solution may be undertaken for a duration of from about 20 seconds to 240 seconds for example 20 seconds, 40 seconds, 60 seconds, 90 seconds, 120 seconds, 180 seconds and 240 seconds. In some embodiments, the predefined temperature is in a range from about 20° C. to about 70° C. for example about 25° C., about 30° C., about 35° C., about 40° C., about 45° C., about 50° C., or about 55° C. In some embodiments, the temperature does not exceed 60° C.

In some embodiments, the washing step (112) may be undertaken with ratio between volume of the solution containing one or more acids and volume of the chips of about 1:2 to about 20:1 for example 1:1, 2:1, 3:1, 4:1, 5:1, 6:1, 7:1, 8:1, 10:1, 12:1, 15:1 and 20:1. In some embodiments, the ratio is about 4:1.

In an exemplary embodiment, the washing step (112) is one cycle of washing by submerging the decellularized chips in hydrochloric acid solution of 0.1 M to 1 M and agitate the slurry for 20-180 seconds, with temperature of the fluid ranging from 20-60° C. The ratio of fluid volume to chip volume is adjusted to 2:1 to 8:1. In another exemplary embodiment, the washing step (112) is one cycle of washing by submerging the chips in hydrochloric acid solution of 0.2 M to 0.7 M and agitate the slurry for 20-60 seconds, with temperature of the fluid not exceeding 60° C. and the ratio of fluid volume to chip volume of 4:1.

Following the contacting of the chips with the acid solution above, the washing step (112) may include a cleaning step to remove any excess of the acid solution. In some embodiments, the cleaning step may include washing the root portion in water or other suitable solvent. In some embodiments, the water may be Type I water, Type II water or water for injection. In some embodiments, the washing may be undertaken at a temperature in a range from about 20° C. to about 60° C. for example about 25° C., about 30° C., about 35° C., about 40° C., about 45° C., about 50° C., or about 55° C. In some embodiments, the washing temperature does not exceed 60° C. It is to be appreciated that the washing temperature may be constant throughout the contacting step or it may be varied. In some embodiments, the washing is undertaken in a shaker. However, it is to be appreciated that other stirring or agitation method including the use of ultrasonic stirring or shaker and magnetic stirrer may also be used. The frequency of the ultrasound used may be in the range from 10 to 60 kHz.

In some embodiments, the washing with water may be undertaken for a duration of from about 1 minute to about 20 minutes, for example 2 minutes, 3 minutes, 4 minutes, 5 minutes, 6 minutes, 10 minutes, 12 minutes, or 15 minutes.

In some embodiments, the washing with water may be undertaken with ratio between volume of water and volume of the chips of about 1:2 to about 20:1 for example 1:1, 2:1, 3:1, 4:1, 5:1, 6:1, 7:1, 8:1, 10:1, 12:1, 15:1 and 20:1. In some embodiments, the ratio is about 4:1. In an exemplary embodiment, the washing with water is six cycles of washing by submerging the chips in Type I water, under stirring for 20-180 seconds, with temperature ranging from 20-60° C. and the ratio of fluid volume to chip volume is adjusted to 2:1 to 8:1. In another exemplary embodiment, the mixture is stirred for about 20-180 seconds, with temperature not exceeding 60° C. and the ratio of fluid volume to chip volume is adjusted to 4:1.

In accordance with some embodiments of the present disclosure, the method (100) described herein may further include lyophilizing (114) the decellularized one or more root portion of the tooth under a predefined lyophilization condition. It is to be appreciated that lyophilization, also known as freeze-drying, is a process of removing water or moisture from a substance, in the present case chips of the decellularized one or more root portion of the tooth by first freezing the chips and then subjecting the chips to a substantially vacuum environment. In some embodiments, the removal of water or moisture via lyophilization (114) may not damage structure or degrade functionality of the chips. In some embodiments, alteration of the structure or functionality may be minimal.

In some embodiments, the lyophilization step (114) may include the steps of freezing (114-i), primary drying (114-ii), and secondary drying (114-iii). In some embodiments, the freezing stage comprising freezing the chips to a very low temperature such that water that may be present in the chips is converted into ice crystals. In some embodiments, the freezing temperature is from about-40° C. to about-80° C. In some embodiments, the freezing temperature is from about-35° C. to about-45° C. In some embodiments, the duration for freezing is about 4 or more hours for example 4 hours, 6 hours, 8 hours and 10 hours. In some embodiments, the minimum duration for freezing is about 4 hours. In some embodiments, as soon as the desired or set temperature or range of temperature is attained (for example −35° C. to about −45° C.), the primary drying step may commence.

Following the freezing step, in the primary drying stage, the frozen chips may be placed in a vacuum chamber, and the pressure in the chamber is lowered to allow the ice to sublimate directly from the solid phase to a vapor without passing through a liquid phase. Consequently, in some embodiments, the primary drying stage removes substantial amount of the water from the chips, leaving behind the dry solid chips. In some embodiments, the primary drying stage may include increasing the temperature to between 15° C. and 30° C. or a room temperature. In some embodiments, the temperature may be increased in gradual manner using constant or variable ramp or step. In some embodiments, the primary drying step may include reducing the pressure to a range from about 0.01 mbar to about 0.8 mbar. In some embodiments, the pressure is reduced to a range from about 0.1 mbar to about 0.5 mbar. In some embodiments, the pressure may be reduced in gradual manner using constant or variable ramp or step. In some embodiments, the primary drying stage may be undertaken for a period of from about 120 minutes to about 480 minutes. In some embodiments, the duration of the primary drying stage may be from about 340 minutes to about 380 minutes.

In some embodiments, at the end of the primary drying stage, the secondary drying stage commences where residual water is removed from the chips. In some embodiments, the removal of residual water is achieved by raising the temperature and lowering the pressure in the vacuum chamber to further promote sublimation. In some embodiments, the removal of residual water is achieved by raising the temperature in the vacuum chamber to further promote sublimation. In some embodiments, the secondary drying stage may include increasing the temperature to a range from about 30° C. and about 60° C. In some embodiments, the temperature is increased to a range from about 30° C. and about 55° C. In some embodiments, the temperature is increased to a range from about 30° C. and about 50° C. In some embodiments, the temperature may be increased in gradual manner using constant or variable ramp or step. In some embodiments, the secondary drying stage may include decreasing the pressure to about 0.05 mbar to about 0.4 mbar. In some embodiments, the secondary drying stage may be undertaken for a period of from about 100 minutes to about 480 minutes. In some embodiments, the duration of the secondary drying stage may be from about 100 minutes to about 140 minutes.

In some embodiments, while lyophilization step (114) may be substituted by other suitable methods of preservation including air-drying or dehydration, lyophilization (114) may preserve the structure and function of the chips by maintaining their molecular and biological integrity. In some embodiments, the lyophilized chips may also have an extended shelf life and increased stability, which can reduce the need for refrigeration or other special storage requirements.

In some embodiments, following lyophilization step (114), the method (100) may optionally include further sterilization (116). In some embodiments, said further sterilization is a second sterilization step. It is therefore to be appreciated that sterilization (106) described above may be considered as the first sterilization step. In some embodiments, unlike the first sterilization step (106), the second sterilization step (116) may be undertaken more than one cycle that is it may be repeated with the same or different set of parameters.

In some embodiments, the second sterilization step comprises sterilizing using one of the following gases: ethylene oxide gas, vaporized hydrogen peroxide, chlorine dioxide gas, vaporized peracetic acid and nitrogen dioxide. In some embodiments, concentration of the gas used during the sterilization is in the range of from about 250 mg/L to about 1,200 mg/L for example 250 mg/L, 300 mg/L, 350 mg/L, 400 mg/L, 450 mg/L, 500 mg/L, 550 mg/L, 600 mg/L, 700 mg/L, 750 mg/L, 800 mg/L, 900 mg/L, 1,000 mg/L or 1,100 mg/L. In some embodiments, the concentration of the gas used during the sterilization is in the range of from about 300 mg/L to about 1,000 mg/L. In some embodiments, the concentration of the gas used during the sterilization is in the range of from about 350 mg/L to about 550 mg/L. In some embodiments, the concentration of ethylene oxide gas used during the sterilization is in the range of from about 300 mg/L to about 1,000 mg/L. In some embodiments, the concentration of ethylene oxide gas used during the sterilization is in the range of from about 350 mg/L to about 550 mg/L.

In some embodiments, the second sterilization step (116) comprises sterilizing using a gamma sterilization or an electron beam sterilization. In some embodiments, the radiation dose is in the range of from about 5 kGy to about 70 kGy for example 5 kGy, 10 kGy, 15 kGy, 20 kGy, 25 kGy, 30 kGy, 35 kGy, 40 kGy, 50 kGy, 55 kGy, 60 kGy or 70 kGy. The unit “Gy” or “Gray” is understood by a person skilled in the art as the absorption of one joule of radiation energy per kilogram of matter (J/kg). In some embodiments, the second sterilization step (116) using Gamma radiation is performed in the range from about 15 kGy to about 25 kGy. In some embodiments, the second sterilization using electron beam radiation is performed in the range from about 15 kGy to about 50 kGy.

In some embodiments, the second sterilization step (116) may be performed more than one cycles as necessary, including two, three, four, five, six and seven cycles. In an exemplary embodiment, the second sterilization step (116) is repeated once, firstly using ethylene oxide followed by gamma or e-beam radiation. In another exemplary embodiment, the second sterilization step (116) is repeated once, both cycles using ethylene oxide. In another exemplary embodiment, the second sterilization step (116) is repeated once, both cycles using gamma or e-beam radiation.

In accordance with some embodiments of the present disclosure, there is provided a graft material from a tooth obtained by a method as described herein. In some embodiments, the graft material is tissue graft material including soft and hard tissues. In some embodiments, the tissue graft material is a periodontal tissue. In some embodiments, the graft material is bone graft material. In some embodiments, the tooth is a human tooth or an animal tooth. In some embodiments, said graft material from the tooth is obtained by the method comprising the steps of providing one or more root portion of the tooth, contacting the one or more root portion of the tooth with a first reagent at a predefined temperature for a predefined duration so as to disinfect the one or more root portion of the tooth and sterilizing the disinfected one or more root portion of the tooth at a predefined sterilization temperature and pressure, thereby obtaining said graft material.

In some embodiments, the graft material as described herein comprises calcium phosphate. In some embodiments, the graft material comprises crystalline or amorphous calcium phosphate. In some embodiments, the graft material comprises hydroxyapatite Ca₁₀(PO₄)₆(OH)₂, octa calcium phosphate or OCP, beta tri calcium phosphate or B-TCP, dicalcium phosphate dihydrate (DCPD) and amorphous calcium phosphate. In some embodiments, the graft material comprises at least 20% (v/v) of octa calcium phosphate or OCP for example 25%, 30%, 40%, 45%, 50%, 55%, 60%, 65%, 70%, 75% or 80% of octa calcium phosphate or OCP. In some embodiments, the graft material comprises from about 65% to about 75% of OCP. In some embodiments, hydroxyapatite in the graft material does not exceed 30%. In some embodiments, hydroxyapatite in the graft material does not exceed 25%. In some embodiments, hydroxyapatite in the graft material preferably does not exceed 20%. In some embodiments, the graft material comprises about 30% to 85% hydroxyapatite. In some embodiments the graft material comprises 30%, 35%, 40%, 45%, 50%, 55, 60%, 65%, 70%, 75% or 80% of hydroxyapatite.

In accordance with some embodiments of the present disclosure, there is provided a graft material from a tooth. In some embodiments, the graft material comprises calcium phosphate. In some embodiments, the graft material comprises crystalline or amorphous calcium phosphate. In some embodiments, the graft material comprises hydroxyapatite Ca₁₀(PO₄)₆(OH)₂, octa calcium phosphate or OCP, beta tri calcium phosphate or B-TCP, dicalcium phosphate dihydrate (DCPD) and amorphous calcium phosphate. In some embodiments, the graft material comprises at least 20% (v/v) of octa calcium phosphate or OCP for example 25%, 30%, 40%, 45%, 50%, 55%, 60%, 65%, 70%, 75% or 80% of octa calcium phosphate or OCP.

As mentioned above, the method described in the present disclosure such as method 100 may retain bioactive materials and bioactive molecules in the graft material. In some embodiments, the bioactive materials comprise bioactive proteins. In some embodiments, the bioactive molecules comprise bioactive proteins. In some embodiments, the bioactive molecules include but are not limited to non-collagenous proteins (NCP) and non-phosphorylated proteins. In some embodiments, non-collagenous proteins (NCP) include but are not limited to Small Integrin-Binding LIgands, N-linked Glycoproteins (abbreviated as SIBLINGs), Small Leucine-Rich Proteoglycans (SLRPs) and matrix phospholipids. In some embodiments, non-phosphorylated proteins include but are not limited to osteocalcin and Matrix Gla protein (MGP), osteonectin or SPARC protein, SLRPs including decorin, biglycan, lumican and calcium binding proteins for example calmodulin, annexins and nucleobindin.

In some embodiments, the presence of bioactive molecules or bioactive materials including bioactive proteins in the graft material is useful as these proteins may promote regeneration of tissue and bone, in particular periodontal tissue and bone, when the graft materials are used in tissue and bone regeneration.

In some embodiments, the method for producing the graft material described herein may advantageously maintain the mechanical strength of the graft material. Hence, the materials may withstand mechanical load and preserve the space for the newly formed tissue and bone.

In accordance with some embodiments of the present disclosure, there is provided a method of regenerating bone or tissue in a subject comprising implanting a graft material as described in the present disclosure at the site of bone where damaged or loss occurs. In some embodiments, the method for regenerating bone or tissue may be an in vivo or an in vitro method. In some embodiments, the site is a bone or tissue portion where at least partial degeneration or loss occurs. In some embodiments, bone loss or damage may be due to normal resorption (shrinkage) of bone, trauma of bone, resection (removal) of bone. In some embodiments, the resorption or the resection of the bone may be due to a disease or treatment of that disease, where said disease is cancer or cyst. In some embodiments, tissue loss or damage may be due to periodontal diseases. In some embodiments, the subject is human or animal. In some embodiments, said graft material is obtained or prepared by the process as described hereinabove.

In accordance with some embodiments of the present disclosure, there is provided a method of repairing defects in bone or tissue in a subject comprising implanting a graft material as described in the present disclosure at the site of defect. In some embodiments, the method for repairing defects in the bone or tissue may be an in vivo or an in vitro method. In some embodiments, the site of defect is a bone or tissue portion where at least partial degeneration occurs. In some embodiments, the subject is human or animal. In some embodiments, said graft material is obtained or prepared by the process as described hereinabove.

As used herein, the terms “repair” or “repairing” refers to a process by which damaged or loss tissue is replaced by scar tissue. In some embodiments repair is useful when the damage to the tissue is extensive for regeneration to occur, or when the tissue is incapable of regeneration.

As used herein, the terms “regeneration” or “regenerating” refers to a process by which damaged tissue is replaced by new functional tissue. In some embodiments, regeneration is useful when the damage to the tissue is limited at a certain degree, and thus the cells within the tissue are capable of dividing and proliferating to replace the damaged or loss tissue. In some embodiments, the damage to the tissue is partial damage and is not to a full extent. In some embodiments, regeneration may result in partial or complete restoration of the tissue to its original state and function.

In some embodiments, there is provided a graft material as described in the present disclosure for use in repairing a defect in bone or tissue in a subject at the site of defect. In some embodiments, there is provided a graft material as described in the present disclosure for use in regenerating bone or tissue in a subject at the site of defect. In some embodiments, the site of defect is part of the bone or tissue where damage occurs.

In some embodiments, said partial degeneration of bone or tissue may be caused by a degenerative bone condition selected from the group consisting of periodontal disease, osteoarthritis, autoimmune diseases (including but not limited to rheumatoid arthritis, Sjogren's syndrome and lupus), osteoporosis, infection, tumors, developmental or congenital disorders, bone cancer and bone disease. In some embodiments, the damaged or loss portion of the bone or tissue may also be caused by trauma or injury, tooth extraction, ill-fitting dentures, bruxism, sinus issues, radiation therapy, and medication (for example administration of bisphosphonates). In some embodiments, the periodontal disease is selected from the group consisting of gingivitis, periodontitis, aggressive periodontitis, chronic periodontitis, systemic periodontitis and necrotizing periodontal disease.

### EXAMPLE

Before explaining at least one embodiment of the disclosure in detail, it is to be understood that the disclosure is not necessarily limited in its application to the details of construction and the arrangement of the components and/or methods set forth in the following Examples. The disclosure is capable of other embodiments or of being practiced or carried out in various ways.

A non-limiting example of processing extracted teeth to the powder graft material is described as follows. The crown is removed from the extracted teeth. For multi-rooted teeth, further root separation is required. After cutting and separation, the crown is discarded and only the root is kept for further use. The roots are submerged in 2.5% sodium hypochlorite in an ultrasonic bath for about 90 minutes, with the temperature of the solution ranging between 40-60° C. The ultrasonic bath frequency is set at 40 kHz. The roots are washed with Type 2 water for about 1 minute to 10 minutes, with the temperature of water ranging between 40-60° C. The roots are submerged in 3-6% hydrogen peroxide in an ultrasonic bath for a duration between 30 minutes to 90 minutes, under temperature ranging between 40-60° C. The ultrasonic bath frequency is set at 40 kHz.

Debris is removed using tungsten carbide bur and further submerged in Type 2 water in an ultrasonic bath for a duration between 30 minutes to 90 minutes at temperature ranging between 40-60° C. The ultrasonic bath frequency is set at 40 kHz. The roots are subsequently autoclaved at a temperature between at 121-134° C. with pressure ranging between 15-30 psi for about 30-60 minutes. Following this step, roots are milled into powder between the range of 200 μm to 950 μm and undergo three cycles of sonication by submerging in 3-6% hydrogen peroxide in Type 1 water with the ratio of fluid to powder between 2:1 to 8:1 for about 10 minutes to 60 minutes with the temperature ranging between 40-60° C. Six to eight washing cycles are performed by submerging the powder in 0.1-1.0 M hydrochloric acid and Type 1 water with manual agitation for about 20 to 180 seconds with the temperature ranging between 40-60° C., using the ratio of fluid to powder at 2:1 to 8:1. Following the washing step above, freezing of the powder is required at a temperature between −80° C. to −40° C. for about 4 to 7 hours before lyophilisation for a duration of about 220-960 minutes at 15-55° C. The final sterilisation is achieved by subjecting the powder in ethylene oxide gas or gamma sterilisation at 300-1000 mg/L or 15-25 kGy, respectively. The powder produced from this process may be used for periodontal regeneration procedures.

It should be appreciated that the above-described methods and materials may be varied in many ways, including omitting, or adding elements or steps, changing the order of steps and the reagents or chemicals used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by a person skilled in the art that the present disclosure is not limited to what has been particularly shown and described hereinabove. Rather the scope of the present disclosure is defined by the claims, which follow.

1. A method of fabricating a graft material from a tooth, comprising:
providing one or more root portions of the tooth only;
contacting the one or more root portions of the tooth with a first reagent comprising sodium hypochlorite or calcium hypochlorite at a predefined temperature for a predefined duration the effective to provide a disinfected one or more root portions of the tooth; and
sterilizing the disinfected one or more root portions of the tooth at a predefined sterilization temperature and pressure, thereby obtaining said graft material.

2. The method of claim 1, further comprising reducing a particle size of the sterilized one or more root portions of the tooth.

3. (canceled)

4. (canceled)

5. The method of claim 1, wherein the first reagent further comprises sodium hydroxide.

6. The method of claim 1, wherein the predefined sterilization temperature is from about 110° C. to about 150° C. and the predefined sterilization pressure is from about 10 psi to about 50 psi.

7. The method of claim 6, wherein the predefined sterilization temperature is from about 121° C. to about 150° C.

8. The method of claim 1, wherein said graft material is suitable for use as a tissue graft material or a bone graft material.

9. The method of claim 1, wherein the contacting to provide a disinfected one or more root portions of the tooth further comprises contacting with a second reagent.

10. The method of claim 9, wherein the second reagent is an oxidizing agent comprising a compound having two oxygen atoms, wherein said two oxygen atoms are linked via a single covalent bond.

11. The method of claim 10, wherein said compound having two oxygen atoms is a peroxide selected from the group consisting of: hydrogen peroxide, peroxy acid, metal peroxide and organic peroxide.

12. The method of claim 2, wherein the reduced particle size of the sterilized one or more root portions of the tooth is from about 100 μm to about 5,000 μm.

13. The method of claim 9, further comprising disintegrating one or more cells from the one or more root portions of the tooth by contacting the one or more root portions of the tooth with the second reagent under ultrasonic energy.

14. The method of claim 13, further comprising washing the decellularized one or more root portions of the tooth with one or more acids having a predefined volume ratio between the one or more acids the root portions of the tooth.

15. The method of claim 14, wherein the predefined volume ratio is in a range from 1:2 to 20:1.

16. The method of claim 13, further comprising lyophilizing the decellularized one or more root portions of the tooth under a predefined lyophilization condition.

17. The method of claim 16, further comprising a second sterilization step.

18. The method of claim 17, wherein the second sterilization step comprises use of a gas selected from the group consisting of ethylene oxide gas, vaporized hydrogen peroxide, chlorine dioxide gas, vaporized peracetic acid and nitrogen dioxide.

19. The method of claim 17, wherein the second sterilization step comprises a gamma sterilization or an electron beam sterilization.

20. A graft material from a tooth, obtained by the method of claim 1.

21. The graft material of claim 20, wherein said tooth is a human tooth or an animal tooth.

22. The graft material of claim 20, wherein the graft material comprises 30-85%(v/v) of hydroxyapatite.

23. A graft material derived from one or more root portions of a tooth, the graft material comprising 30-85%(v/v) of hydroxyapatite.

24. A method of regenerating bone or tissue in a human, comprising implanting the graft material of claim 20 into a damaged or lost portion of the bone or tissue.

25. The method of claim 24, wherein said damaged or lost portion of the bone or tissue is caused by a disease selected from the group consisting of: periodontal disease, osteoarthritis, autoimmune diseases, osteoporosis, infection, tumors, developmental or congenital disorders, bone cancer and bone disease or by a condition or treatment selected from the group consisting of trauma or injury, tooth extraction, ill-fitting dentures, bruxism, sinus issues, radiation therapy, and administration of bisphosphonates.

26. The method of claim 25, wherein the periodontal disease is selected from the group consisting of: gingivitis, periodontitis, aggressive periodontitis, chronic periodontitis, systemic periodontitis and necrotizing periodontal disease.
